(19) United States
(12) Patent Application Publication
(10) Pub. No.: US 2024/0285232 A1
(43) Pub. Date: Aug. 29, 2024

(54) DEVICE AND SOFTWARE APPLICATION FOR DETECTING BLOOD ALCOHOL CONTENT

(71) Applicant: Tracklet Inc., Huntington Beach, CA (US)

(72) Inventors: John Stuart Rance, Huntington Beach, CA (US); Jose Aaron Gonzalez, McAllen, TX (US)

(73) Assignee: Tracklet Inc., Huntington Beach, CA (US)

(21) Appl. No.: 18/589,334

(22) Filed: Feb. 27, 2024

Related U.S. Application Data
(60) Provisional application No. 63/448,659, filed on Feb. 27, 2023.

(51) Int. Cl. A61B 5/00; A61B 5/11; A61B 5/145; G16H 40/67; G16H 50/30

(52) U.S. Cl. CPC A61B 5/4845; A61B 5/1112; A61B 5/14517; A61B 5/681; A61B 5/7264; A61B 5/7275; A61B 5/746; G16H 40/67; G16H 50/30

(57) ABSTRACT

Embodiments of the invention include devices, systems, methods and software applications that automatically detect measured blood alcohol content (BAC). In aspects, a blood alcohol content sensor detects blood alcohol content by measuring an amount of sweat via a wearable device. The systems and methods can also predict the future BAC of a subject by considering the present BAC along with physiological events and/or historical data. The results can be reported to a server to applying a protocol in response to the reported results.

### FIELD OF THE INVENTION

The invention relates to devices, systems, methods and software applications that automatically detect measured blood alcohol content (BAC) and report the results to a server.

### BACKGROUND

Alcohol intoxication and its subsequent impairment results in myriad deleterious effects to those involved both directly and indirectly. Not only does alcohol abuse present the potential for death, damages, and harm, it is also a drain on the economy. According to the Centers for Disease Control (CDC), an estimated $28 billion in healthcare expenses are attributable to alcohol abuse and impairment, as well as $179 billion in lost workplace productivity. Another $13 billion in economic cost is calculated as the result of automobile accidents as well as $25 billion in criminal justice expenditures. Drunk driving is one of a series of dangerous activities that manifest from alcohol impairment, often resulting in legal and lethal consequences.

Alcohol is a drug that impairs the human body across several dimensions of physiology and psychology. It impairs hand-eye coordination as well as impairing mental processes such as reasoning, hampering the ability to make informed decisions. Impairment due to alcohol can lead to consequences that endanger not only the user, but also those around them. Elevated alcohol consumption has long been known as a factor in domestic abuse, workplace harassment, and repeated failures to meet responsibilities and obligations. Alcohol abuse can destroy personal lives and dismantle families, putting many abusers on a path of repercussions they are unable to resolve until abstinence from alcohol is achieved.

Alcohol is primarily absorbed through the walls of the stomach and small intestine. This absorption results in increased blood alcohol content (BAC). A level at or exceeding 0.08% BAC is above the legal limit for operating vehicles and heavy machinery in most states and municipalities. Intoxication at levels above the typically mandated legal limit of 0.08% BAC can lead to severe physiological and psychological consequences. At levels around 0.10% BAC, speech tends to be slurred as reaction time and thought processing is slowed. At levels around 0.15% BAC, many users experience an alteration in mood, in addition to loss of balance, muscle control and increased chance of nausea and vomiting. At levels above 0.15% BAC up to 0.30% BAC, it is likely for a user to experience the life-threatening condition of alcohol poisoning which may result in a loss of consciousness and possibly death. Levels of 0.40% BAC and above can be fatal due to respiratory arrest and often requires immediate and acute care.

Alcohol abuse may also lead to alcohol dependence. Drinkers can become physiologically dependent on alcohol and experience symptoms of withdrawal. To overcome withdrawal symptoms, users tend to imbibe more alcohol and do so more frequently, leading to an alteration of habits and lifestyle choices which makes it more difficult for a dependent user to perform their expected tasks and fulfill their responsibilities.

A longtime issue has existed in the workplace relating to the abuse of alcohol. For employers who rely upon a workforce with mission-critical job duties, abusing alcohol can carry serious and severe consequences, not only for the employee, but also for the employer and the company. Such consequences include, for example, missed assignments, missed appointments, inaccurate or incomplete work, delays in duty fulfillment, workplace truancy, insubordination and negligent/criminal behavior.

Companies with chronically intoxicated employees often lose out on profit potential while setting themselves up for liability. In certain circumstances, these are unforeseen and unpredictable liabilities due to the lack of knowledge regarding an employee's addiction to alcohol. This withholding of information affects all industries. For example, if an insurance provider is denied information regarding a client's alcohol consumption, then that provider is not able to accurately assess the appropriate risk of a client, and therefore may issue policies that are unable to cover their costs should an alcohol-related incident occur.

A chronically intoxicated employee is typically an unreliable performer across the spectrum of workplace responsibilities and is frequently a physical and psychological danger to those around them. Colloquially known as “functional alcoholics,” these members of a workforce may hide their addiction from those who hire them, but often cannot maintain their commitments over the long term and can eventually succumb to deeper levels of addiction. Presenting an unforeseen liability to a company and its employees which is only mitigated by employment termination and in certain instances criminal prosecution.

Blood alcohol testing can be used to address problems associated with drinking. The most accurate way to test is to withdraw a blood sample for testing in a laboratory. However, blood testing is impractical because it is intrusive, expensive and may require hours (or days) to get a result. Portable breathalyzer systems have been used by law enforcement and are generally considered the primary means available for field testing. However, they are typically conspicuous, cumbersome and intrusive to use. What is needed is a wearable detection system that is non-intrusive and inconspicuous.

Therefore, an unmet need exists in the art to track and report the blood alcohol content of individuals who are expected to reliably perform workplace duties and responsibilities in which alcohol is an impairment. In addition, an unmet need also exists for tracking those who are court-mandated to follow specific sobriety guidelines so that they can rehabilitate and no longer present a danger to society.

### SUMMARY OF THE INVENTION

The inventions described and claimed herein have many attributes and embodiments including, but not limited to, those set forth or described or referenced in this brief summary. The inventions described and claimed herein are not limited to, or by, the features or embodiments identified in this summary, which is included for purposes of illustration only and not restriction.

Embodiments include a device and software system which can collect blood alcohol concentration (BAC) data, send it to computer/mobile device (APP), and onto a server which applies a protocol that can activate an alert or render advice/instructions.

In embodiments, a blood alcohol content (BAC) is determined by sampling a small amount of sweat from a user via wristwatches, bracelets, smartwatches or other equivalent wearable devices that are known in the art. These devices communicate with a series of mobile devices, databases, computing systems, IP switches, servers and other equivalent structures and storage devices that are known in the art.

Embodiments include a computer-implemented method for monitoring and predicting blood alcohol levels in a subject. The method can include steps of (a) detecting a present blood alcohol content using one or more sensors on a wearable device, (b) estimating a future blood alcohol content, (c) reporting the present blood alcohol content and the future blood alcohol content to a server and (d) activating an alert system if the present blood alcohol content and/or the future blood alcohol content is at or above a threshold level.

In embodiments, the computer-implemented method also includes a step of detecting one or more physiological events in the subject (via sensors in the wearable device). The physiological events can include, for example, eye/pupil activity, heart rate, change in heart rate, bodily movement, temperature, change in temperature, blood pressure, change in blood pressure, respiration rate, level of oxygen saturation, perspiration rate and/or blood glucose level. The events can be used in predicting the future blood alcohol content of the user. In aspects, the method also utilizes historical data to predict a future blood alcohol level of the user.

In aspects, the future blood alcohol content is estimated at about 15 to 120 minutes from the present time. In aspects, an alert system is activated when either the present or the future predicated blood alcohol content is 0.08%.

In aspects, the method uses deep learning and/or a neural network in the step of evaluating and predicting impairment. In aspects, the method uses a data model selected from a linear regression model, a polynomial regression model, a naïve Bayes model and a gradient boosted model.

In embodiments, sensors in the wearable device detect the present blood alcohol content using body sweat of a wearer (i.e., transdermal detection).

In embodiments, the method includes an alert system that notifies one or more of a parent, employer, probation officer, insurance company, ride share agent, tow truck operator, group leader or custodial agent. The alert system can also include geographical location information of a user.

Embodiments also include a computer-implemented method for evaluating and predicting impairment in a subject. The method can include steps of (a) detecting a blood alcohol content using one or more sensors on a wearable device, (b) detecting one or more physiological events using one or more sensors on a wearable device, (c) determining a present level of impairment based on the blood alcohol content and the one or more physiological events, (d) estimating a future level of impairment based on the blood alcohol content and the one or more physiological events, (e) reporting the present level of impairment and future level of impairment to a server and (f) activating an alert system if the present level of impairment and/or the future level of impairment is at or above a threshold level. In aspects, the method also utilizes historical data to estimate a future level of impairment.

Embodiments also include a smartphone application (APP) configured to (a) communicate with a wearable device of a user, (b) retrieve a blood alcohol content of the user via an alcohol sensor within the wearable device (via sweat), (c) detect one or more physiological events using a sensor on the wearable device, (d) create a data model based on the blood alcohol content of the user and (e) predict a future blood alcohol content of the user for the next 15 to 120 minutes.

In aspects, the APP uses a linear regression model, a polynomial regression model, a naïve Bayes model or a gradient boosted data model. A wearable device can have sensors that are configured to detect the user's current temperature, compare the user's current temperature to a baseline temperature and automatically calculate the user's BAC from the comparison of the two temperatures. In aspects, the methods described herein include a step of encryption.

The APP can also be configured to (a) determine if the blood alcohol content or the future blood alcohol content is above a threshold, (b) report the blood alcohol content and/or the future blood alcohol content to a receiving party, (c) prompt the user via one of SMS message, iMessage, Android message, or VoIP to remain at a location and (d) engage a rideshare service to report to the location of the user.

In embodiments, the invention includes an NFC antenna and/or a Bluetooth® module integrated into a wearable device such as a bracelet or wristwatch. The NFC antenna and Bluetooth® module can communicate with software on a mobile phone application and an operating system within the mobile phone to deliver and receive data packets and applicable information to and from the Internet and servers via an IP switch and Internet gateway. In aspects, the invention can communicate with a database and is compatible with integrated voice response, SMS messaging, VoIP messaging, and other equivalents that are known in the art.

Other features and advantages of aspects of the present invention will become apparent from the following more detailed description, taken in conjunction with the accompanying drawings, which illustrate, by way of example, the principles of aspects of the invention.

### BRIEF DESCRIPTION OF THE DRAWINGS

The accompanying drawings illustrate aspects of the present invention. In such drawings:

FIG. 1 displays a sample user login screen which prompts an end user for their username and password.

FIG. 2 is an algorithmic flow chart of an embodiment of the protocol applied within the present invention.

FIG. 3 is a flow chart of an embodiment of the interconnection of communicating devices within the systems of the present invention.

### DEFINITIONS

Reference in this specification to “one embodiment/aspect” or “an embodiment/aspect” means that a particular feature, structure, or characteristic described in connection with the embodiment/aspect is included in at least one embodiment/aspect of the disclosure. The use of the phrase “in one embodiment/aspect” or “in another embodiment/aspect” in various places in the specification are not necessarily all referring to the same embodiment/aspect, nor are separate or alternative embodiments/aspects mutually exclusive of other embodiments/aspects. Moreover, various features are described which may be exhibited by some embodiments/aspects and not by others. Similarly, various requirements are described which may be requirements for some embodiments/aspects but not other embodiments/aspects. Embodiment and aspect can in certain instances be used interchangeably.

The terms used in this specification generally have their ordinary meanings in the art, within the context of the disclosure, and in the specific context where each term is used. Certain terms that are used to describe the disclosure are discussed below, or elsewhere in the specification, to provide additional guidance to the practitioner regarding the description of the disclosure. It will be appreciated that the same thing can be said in more than one way.

Consequently, alternative language and synonyms may be used for any one or more of the terms discussed herein. Nor is any special significance to be placed upon whether or not a term is elaborated or discussed herein. Synonyms for certain terms are provided. A recital of one or more synonyms does not exclude the use of other synonyms. The use of examples anywhere in this specification including examples of any terms discussed herein is illustrative only, and is not intended to further limit the scope and meaning of the disclosure or of any exemplified term. Likewise, the disclosure is not limited to various embodiments given in this specification.

Without intent to further limit the scope of the disclosure, examples of instruments, apparatus, methods and their related results according to the embodiments of the present disclosure are given below. Note that titles or subtitles may be used in the examples for convenience of a reader, which in no way should limit the scope of the disclosure. Unless otherwise defined, all technical and scientific terms used herein have the same meaning as commonly understood by one of ordinary skill in the art to which this disclosure pertains. In the case of conflict, the present document, including definitions, will control.

The term “blood alcohol level” or “BAC” refers to the percent of alcohol (ethyl alcohol or ethanol) in a person's blood stream. A BAC of 0.10% means that an individual's blood supply contains one part alcohol for every 1000 parts blood. Blood alcohol levels are usually determined in breath and blood samples. The principle of BAC measurement is based on the fact that alcohol, taken orally, goes into the body system. Equilibrium distribution of alcohol into the different parts of the body mainly liver, kidney, brain, and lungs is attained very rapidly. The ratio of alcohol in the blood to alcohol in alveolar air is approximately 2,100:1 at 34° C., the temperature at which the breath leaves the mouth. Thus, the extent of alcohol intoxication or alcohol consumption is monitored by examining BAC in breath and blood of the affected person, but the obvious choice is blood, an absolute level can be obtained only by drawing a sample of blood. There are several methods for the estimation of BACs using iodometric titrations, breath analyzer, and biosensors. For the purposes of the present invention and clarity of disclosure, “Blood Alcohol Content,” “Blood Alcohol Concentration,” “Blood Alcohol Level” and “Blood Alcohol Volume” are treated as equivalent and synonymous.

The term “physiological event” refers to a response or reaction of the body to a stimulus. Most are automatic/instinctive physiological responses. The healthy state of the body depends upon the integrity of various organ systems. The organ systems in the body function in a particular manner constantly. The mechanisms by which the organ systems of the body function, can be referred to as “physiological mechanisms.” Physiological mechanisms explain any health-related events or outcomes.

Physiological mechanisms can be altered voluntarily. For example, exercise causes alteration (i.e., stimulation) in the cardiac physiology of resting state. Similarly, a central nervous system (CNS) depressant such as alcohol slows down parts of the brain and results in impaired cognitive function. Physiological events can include, for example, eye/pupil activity, heart rate, change in heart rate, bodily movement, temperature, change in temperature, blood pressure, change in blood pressure, respiration rate, level of oxygen saturation, perspiration rate and blood glucose level.

The term “historical data” refers to data collected about past events and circumstances pertaining to a particular subject. It can include, for example, social habits, physiological responses to alcohol, changes in impairment levels, etc.

The term “impairment” or “functional impairment” refers to a difficulty in taking care of oneself or performing routine tasks/actions. For example, impairments can be vision, hearing or movement limitations or growth, behavioral, speaking or physical. Impairment can be quantified on a scale of 0 to 100:0 to 4 percent is interpreted as no problem, 5 to 24 percent is a mild problem, 25 to 49 percent is a moderate problem, 50 to 95 percent is severe, and 95 to 100 is total or complete problem. “Impairment” can also refer to any number of conditions that may reduce or negatively affect a one's abilities to operate a motor vehicle. They can include drowsiness, fatigue, distraction, intoxication, illness, anxiety or agitation.

The term “data fusion system” refers to a system that can align/integrate data sets and combine them to produce a meaningful result or conclusion.

The term “fusion” or “data fusion” refers to the process of integration of multiple data and knowledge representing the same real-world object into a consistent, accurate, and useful representation.

The term “high-level fusion” refers to the ability of a system to capture awareness and complex relations, reason over past and future events, utilize direct sensing exploitations and tacit reports, and discern the usefulness and intention of results to meet system-level goals.

The term “wearable device” or “wearable technology” refers to devices that a user can attach to their body to collect health and fitness data, which they may provide to doctors, health providers, insurers and other relevant parties. Examples include fitness trackers, blood pressure monitors and biosensors. Smartwatches, wristbands, smart shoes and smart jewelry are also examples of wearable technology.

A wearable device can include various sensors, for example, body posture sensors, audio sensors, motion sensors, heart rate sensors and physiologic sensors. Other sensors that can monitor physiological activity of a wearer include: heart/pulse rate, blood oxygen levels, blood pressure, respiration rate/pattern, heart rate variability, body posture, body movement, electromyographic data, head position, head movement, eye direction, eye movement, gaze pattern/direction, eyelid opening, blink rate, eyebrow activity, pupil size, pupil activity, facial expressions, facial activity, speaking activity, speaking volume. The system can also include sensors to detect substances in a subject's system such as alcohol, drugs and/or drug metabolites (via transdermal analysis of body sweat).

The term “IP Switching” refers to a methodology for routing data packets over the network that does the following: routes data packets as typical routers do and employs switching mechanisms for the rapid transfer of data. The term “IP switch” refers to an IP (internet protocol) router with attached switching hardware that has the ability to cache routing decisions in switching hardware. To construct an IP switch, a standard ATM (Asynchronous Transfer Mode) switch is taken, the hardware is left untouched, but all the control software above AAL-5 is removed. It is replaced by standard IP routing software, a flow classifier to decide whether to switch a flow or not and a driver to control the switch hardware. At system startup a default virtual channel is established between the control software of the IP switch and its neighbors, which is then used for default hop-by-hop forwarding of IP datagrams. To gain the benefits of switching, a mechanism has been defined to associate IP flows with the ATM labels.

As used in this application, and unless the context dictates otherwise, the term “coupled to” is intended to include both direct coupling (in which two elements that are coupled to each other contact each other) and indirect coupling (in which at least one additional element is located between the two elements). Therefore, the terms “coupled to” and “coupled with” are used synonymously.

The term “encryption” refers to a process of encoding information. This process converts the original representation of the information, known as plaintext, into an alternative form known as ciphertext. Ideally, only authorized parties can decipher a ciphertext back to plaintext and access the original information. Encryption does not itself prevent interference but denies the intelligible content to a would-be interceptor. For technical reasons, an encryption scheme usually uses a pseudo-random encryption key generated by an algorithm.

It should be noted that any language directed to a computer should be read to include any suitable combination of computing devices, including servers, interfaces, systems, databases, agents, peers, engines, controllers, or other types of computing devices operating individually or collectively. One should appreciate that the computing devices comprise a processor configured to execute software instructions stored on a tangible, non-transitory computer readable storage medium (e.g., hard drive, solid state drive, RAM, flash, ROM, etc.). The software instructions preferably configure the computing device to provide the roles, responsibilities, or other functionality as discussed below with respect to the disclosed apparatus. In embodiments, the various servers, systems, databases, or interfaces exchange data using standardized protocols or algorithms, possibly based on HTTP, HTTPS, AES, public-private key exchanges, web service APIs, known financial transaction protocols, or other electronic information exchanging methods. Data exchanges preferably are conducted over a packet-switched network, the Internet, LAN, WAN, VPN, or other type of packet switched network. The following description includes information that may be useful in understanding the present invention. It is not an admission that any of the information provided in this application is prior art or relevant to the presently claimed invention, or that any publication specifically or implicitly referenced is prior art.

As used in this application, terms like “set” or “subset” are meant to be interpreted to include one or more items. It is not a requirement that a “set” include more than one item unless otherwise noted. In some contexts, a “set” may even be empty and include no items.

As applicable, the terms “about” or “generally”, as used herein in the specification and appended claims, and unless otherwise indicated, means a margin of +/−20%. Also, as applicable, the term “substantially” as used herein in the specification and appended claims, unless otherwise indicated, means a margin of +/−10%. It is to be appreciated that not all uses of the above terms are quantifiable such that the referenced ranges can be applied.

“SMS” generally refers to an initialism for “Short Message Service,” and is generally defined as a text message sent on a cellular device.

“iMessage” generally refers to a text/instant messaging platform supported by Apple's proprietary operating system, iOS.

“Android message” generally refers to a text/instant messaging platform supported by Google's proprietary operating system available on Google, Android devices and their equivalents and licensees.

“VoIP” generally refers to an initialism for “Voice Over Internet Protocol” and is generally defined as a telecommunications system that uses the Internet or other Internet Protocol networks to transmit telephone calls.

An “IP switch” generally refers to a router which routes Internet protocol traffic over high-speed packet switches (typically Asynchronous Transfer Mode switches) using a proprietary control protocol.

An “Internet gateway” generally refers to another term for a router, specifically when the router is in a local network. Its primary purpose is to forward packets of data to the Internet and receive packets of data from the Internet.

A “packet” generally refers to a unit of data made into a single package that travels along a given network path. Data packets are used in Internet Protocol (IP) transmissions for data that navigates the World Wide Web and also navigates within other networks.

“Near Field Communication” or “NFC” generally refers to communication over very short distances, such as by contact or near-contact of electronic devices.

A “Radio Frequency Identification Chip” or “RFID” generally refers to a computer chip connected to miniature antennas that can be placed on or in physical objects. RFID chips are used in myriad applications where contactless authentication is desired.

“Bluetooth®” generally refers to a standard for the short-range wireless interconnection of mobile devices, computers and other electronic devices.

A “wearable device” generally refers to a near-field communication enabled device that includes without limitation a wristband, wristwatch, smartwatch, ring, or other device that is worn on the user that is used to perform a transaction.

“Mission-critical” or “mission-critical job duties” generally refer to anything that is vital to the functioning of an organization.

“Ride-share services” generally refer to a vehicle or helicopter service that matches drivers of private cars or pilots with people seeking transportation. In contemporary terms, it's an arrangement where passengers connect with drivers or pilots through mobile applications or websites, allowing transportation of the passengers to a certain destination provided by an assigned driver or pilot.

A “gradient boosted model” generally refers to a model derived from machine learning techniques utilized for regression and classification tasks. A gradient boosted model generally allows for prediction in the form of an ensemble (or collection) of weak prediction models (such as decision trees).

A “linear/polynomial regression model” generally refers to a form of linear regression where only due to the non-linear relationship between dependent and independent variables are polynomial terms added to the linear regression to convert the linear regression model into a polynomial regression model.

A “naïve Bayes model” generally refers to a simple technique for constructing classifiers, i.e., models that assign class labels to problem instances, represented as vectors of feature values, where the class labels are drawn from some finite set.

A “smartphone” generally refers to a mobile phone or mobile device with advanced features and computing capacity.

An “end user” generally refers to the final consumer of a product and/or the intended recipient or user of a product.

“Apple iOS” generally refers to a closed and proprietary operating system present on Apple devices and developed by the company Apple.

An “Android operating system” generally refers to a proprietary operating system present on Android devices and developed by the company Google.

A “proprietary operating system” generally refers to an operating system that a particular company conceptualizes, designs, develops and sells.

A “proprietary data base” generally refers to a database that a particular company conceptualizes, designs, develops and sells.

A “proprietary app” or “proprietary application” generally refers to a program (application) that a particular company conceptualizes, designs, develops and sells.

A “server” generally refers to a computer or network of computers that store and provide data to other computers and equivalent devices.

An “IVR” or “interactive voice response” generally refers to an automated telephone system that combines pre-recorded messages or text-to-speech technology with dual-tone multi-frequency (DTMF) interface to engage callers, allowing them to provide and access information without a live agent.

A “public switched telephone network” generally refers to an international telephone system that uses copper wires to carry analog voice data. It consists of a collection of individual telephones that are hardwired to a public exchange.

“Communication Protocol” generally refers to a system of digital message formats and rules for exchanging messages in or between computing systems (e.g., in telecommunications). Protocols may include signaling, authentication, and error detection and correction capabilities. Each message has an exact meaning intended to provoke a defined response by the receiver. The nature of the communication, the actual data exchanged, and any state-dependent behaviors are defined by a technical specification or communication protocol standard. Examples of conventional communication protocols include, without limitation, HTTP, HTTP Secure (HTTPS), File Transfer Protocol (FTP), etc.

The term “user” or “end user” generally refers to any operator of the devices, systems and methods of the present invention or any person or entity the devices, systems and methods of the present invention are designed for. The terms “user” and “end user” may also be interchangeable and synonymous.

Other technical terms used herein have their ordinary meaning in the art that they are used, as exemplified by a variety of technical dictionaries. The particular values and configurations discussed in these non-limiting examples can be varied and are cited merely to illustrate at least one embodiment and are not intended to limit the scope thereof.

### DETAILED DESCRIPTION

It is to be understood that both the foregoing general description and the following detailed description are exemplary and explanatory and are intended to provide further explanation of the subject technology as claimed. Additional features and advantages of the subject technology are set forth in the description below, and in part will be apparent from the description, or may be learned by practice of the subject technology. The advantages of the subject technology will be realized and attained by the structure particularly pointed out in the written description and claims thereof.

The accompanying disclosure provides example embodiments of the inventive subject matter. Although each embodiment represents a single combination of inventive elements, the inventive subject matter is considered to include all possible combinations of the disclosed elements. Thus, if one embodiment comprises elements A, B, and C, and a second embodiment comprises elements B and D, then the inventive subject matter is also considered to include other remaining combinations of A, B, C, or D, even if not explicitly disclosed.

Alcohol abuse encompasses a spectrum of unhealthy alcohol drinking behaviors, ranging from binge drinking to alcohol dependence. In extreme cases, alcohol abuse leads to health problems for individuals and large-scale social problems such as alcohol-related crimes. Even moderate alcohol use can lead to liabilities for individuals and employers. However, alcohol is the most recreationally used drug internationally and is widely available and often socially accepted.

Breathalyzer machines and other systems are commercially available. They have been commonly used to measure the impairment of a driver before or during operation of a vehicle. Some systems have attempted to directly measure a driver's impairment by requiring one to push buttons in a random order within a given amount of time. Other systems have attempted to determine the driver's blood alcohol concentration by measuring tissue alcohol concentration or breath alcohol concentration. However, the complexity, expense and intrusiveness of these systems typically makes them impractical for daily use.

Transdermal ethanol detection has been proposed as a method of preventing drunk driving if integrated into an ignition interlock system. In theory, sensors in a motor vehicle could prevent one from driving if their blood alcohol level was above a threshold (e.g., 0.08%). However, there is a significant delay between alcohol ingestion and detection at the skin. This makes real time estimation of blood alcohol concentration via skin measurement difficult. For example, a dose of 15 ml of ethanol can have a peak lag time of approximately 33 minutes; a dose of 60 ml of ethanol can have a peak lag time of 53 minutes.

Embodiments of the invention include devices, systems and methods for detecting a user's blood alcohol content. The systems and methods can also predict the future BAC of a subject by considering the present BAC along with physiological events and/or historical data.

### Application (APP) Logic

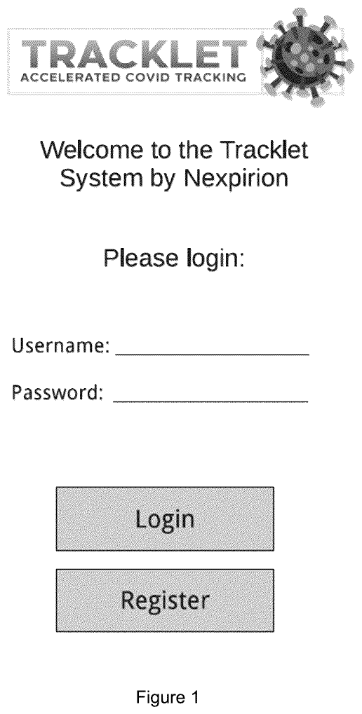

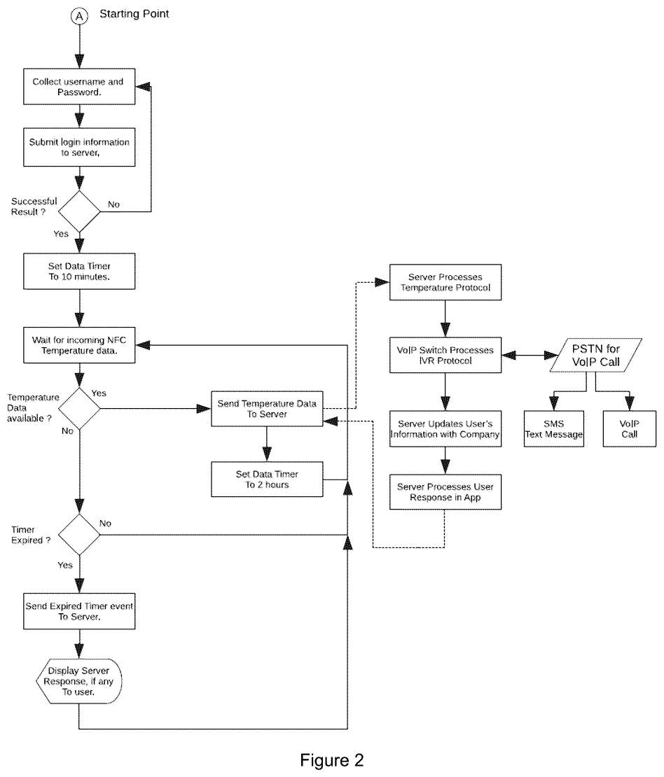

A user begins use by downloading the APP as shown in FIG. 1 and FIG. 2. After the user downloads the APP, they will start by selecting a username and password. Then the user must logon to the APP by entering the username and password. If the user does not have an existing account, the APP will offer a registration screen to establish a new user account. As part of this process, the user's account will be linked to its corresponding corporate account (if any).

Upon the user's attempt to login, the user's login credentials are sent to the server for verification. If the credentials are invalid, the user is prompted with an error message and asked to re-enter the login information. Once the APP verifies the login credentials, it goes into its normal data gathering loop, waiting for an NFC event to occur. The APP has a wait timer which the APP uses to determine if too much time has passed since the last NFC event. Initially, this timer is set to ten minutes after the user logs into the APP.

After a login, the APP waits for the user to place the phone in near proximity to the bracelet or wristband. When the user performs this action, an NFC event occurs and the APP can accept the transfer of the data packet and thereby read the blood alcohol concentration of the user from the data packet. The APP then sends this data to the IP server over the phone's internet connection. The server will then process the data following a protocol selected by the user's company. This protocol will use the data received from the APP, along with the user's historical temperature data retrieved from the database, to determine what action, if any, should be taken.

Under normal circumstances, no action is needed. Thereafter, the server sends a response back to the user's APP acknowledging receipt of the data. The APP sets the wait timer to expire after two hours and waits for the next NFC event or Bluetooth® event. During the server's application of the protocol to the processing results, if the protocol determines that some action is required, the server will simultaneously update the user's company, typically by sending an information record to a company server, as well as alert the user with a text message.

If the action required is messaging the user with additional information, the server sends the message back to the user's APP for display. This message appears on the user's smartphone using the normal IOS or Android facilities for displaying notifications. The server can initiate an SMS (short message service) or text message script through the SMS module. The user will be presented with a series of questions and/or directions specified by the protocol.

If a more immediate connection to the user is required, the server can direct the VoIP gateway to initiate an IVR (interactive voice response)-based VoIP call to the user's smartphone. The user will be presented with a custom set of voice menus specified by the protocol. Once the server receives a response from the user through the APP, the APP will display the message that the protocol selects to send to the user, if any. In the case no NFC event is received before the timer expires, the APP will inform the server that the user is now late in performing a blood alcohol concentration check. The first time this occurs, the server will send back a message reminding the user to perform a blood alcohol concentration check as soon as possible. If this is not performed in a reasonable amount of time, the server will initiate a VoIP call and a SMS message to remind the user about the need to perform a temperature check. When the user logs out of the APP, the server will no longer monitor for temperature data from this user until the next time the user logs back into the APP.

### Method of Monitoring Blood Alcohol Content (BAC)

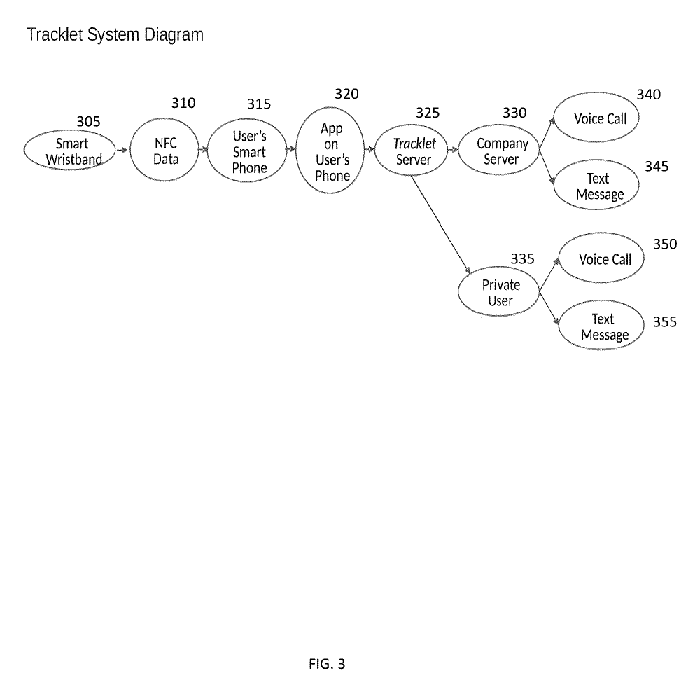

FIG. 3 is a flowchart of the system according to embodiments. A blood alcohol concentration (BAC) sensor device is integrated into a wearable device 305 which is typically worn by the user (e.g., wristband, bracelet or watch). The device can sample a small quantity (e.g., microliters) of sweat and analysis of the BAC. This testing can be done periodically (e.g., every five minutes) or intermittently. Near field communication (NFC) data 310 can be transferred to a smartphone 315. An APP on the smartphone 320 can process and present data and related information. The smartphone can transfer data to a hosted server 325 and a company server 330 and/or a private user 335.

Data obtained by the device is analyzed by the “KnowBAC” system for processing. It can communicate relevant information from the system to the user and/or the user's employer.

The data is taken in by a front end for processing and recorded in a database. The data is fed into a predictive model based on the weight, height and gender data obtained during the registration process. The model is tailored for each specific user over time as more data is obtained.

The model can project what the user BAC will be in the next 15 to 120 minutes. If one of the curves projects a BAC above an employer specified limit (typically 0.08%), the system can send a report to the user's employer and place an alert on the user's smartphone. The employer can be provided with the prediction levels along with the user's current GPS location.

At the same time, the system steps through an employer specified set of actions depending on the BAC level predicted. The set is database driven and include an arbitrary number of decision points. The actions can include a robust set of communication methods through the KnowBAC App, the sending SMS text messages to the employee's phone or the VoIP IVR phone calls. The system can also interface with the employer's own systems to initiate customized actions such as locking out card key credentials, communicating with third-party service providers to send personnel (e.g., taxi cabs, ride share services, limousine services or tow truck operators) to assist the employee at their GPS location. The system can also place the employee in direct VoIP contact with designated medical personnel.

In aspects, the system can use three predictive models (Gradient Boosted Model, Linear/Polynomial Regression, Naive Bayes) to project near-term BAC levels once a rise in BAC is detected.

The invention can also assess blood alcohol content by monitoring the temperature of an end user, utilizing devices including bracelets, wristwatches, smartwatches and other wearable devices that are recognized and well-known in the art. These devices communicate with a series of mobile devices, databases, computing systems, IP switches, servers and other equivalent structures and storage devices that are recognized and well-known in the art.

The data collected by the end user device can be transferred to an application on the end user's smartphone or equivalent mobile device through near field communication (NFC), Bluetooth® or other equivalent and art-recognized communication modality.

The data can be collected by a front-end server for processing and recorded in a database or other equivalent structure. The data is fed into a predictive model based on the user's weight, height, gender and obtained data retrieved during the device registration process. A model is created from this collection of data and is tailored to each specific user. The model can be modified over time as more data is obtained.

For the purpose of monitoring the BAC of an employee, client or group member, the data processed by the system can be relayed to the user's employer, group leader, insurance carrier, probation officer or any other party subject to the signed agreement conveying the end user's informed consent.

The predictive power of the obtained data model allows extrapolation of what an end user's BAC will be within a period of time (e.g., 15 to 120 minutes) of data collection. If one of the data-derived curves projects a BAC above a contractually specified limit (typically 0.08%), then the system can send a report to the end user's employer (i.e., receiving party) and place an alert on the end user's smartphone. The employer can be informed of prediction levels along with the end user's current GPS location. In lieu of an employer, these reports may be sent to a group-leader, insurance carrier, probation officer and any other party subject to the contractual agreement.

For example, the system further guides the employer through a specified set of potential actions depending on the BAC level predicted for the end user. This data set is database driven and includes an arbitrary number of decision points. The actions available to the employer can include a robust set of communication methods through the proprietary app of the present invention, such as SMS text messages, iMessage, Android messaging, VoIP IVR phone calls or any other audio/visual communication-based equivalent (340, 345, 350 and 355). The system can also interface with the employer's own systems to initiate customized actions such as locking out key card credentials and communicating with third party service providers to send personnel such as taxi cabs, ride-share services, limousine services, or tow truck operators to assist the employee at their reported GPS location. The tow truck operators or equivalent personnel can provide a flat-bed truck or other towing system to safely retrieve the employee's vehicle and deposit the vehicle at a preferred location. The system can also place the employee in direct VoIP contact with designated emergency medical personnel.

Predictive models are employed to project and extrapolate BAC levels once a rise in BAC is detected by the system. These predictive models can include models such as a gradient boosted model, a linear/polynomial regression model, and a naïve Bayes model.

Embodiments of the invention also include an IP switch. The IP switch can gather the blood alcohol concentration data and apply a protocol to determine if instructions need to be delivered to the APP on the smartphone of the employee or member of a group.

The system can also collect data related to “physiological events” of a wearer such as body temperature. Accordingly, embodiments include a temperature sensing device or temperature sensing chip incorporated into a wearable device designed to be worn on an end user's wrist. The temperature sensing device or temperature sensing chip can work in coordination with the wearable device and blood alcohol concentration system to track a user's blood alcohol concentration by comparing their BAC to the recorded temperature against a baseline temperature obtained earlier from the user.

A baseline temperature model can be obtained upon user registration and provide a basis for comparing current body temperature data of the user to determine if the current body temperature indicates an abnormal deviation. The data model also can include sufficient data points to predict if a particular individual user is likely to experience an increase in body temperate over a subsequent period of time (e.g., within two to three hours). In addition, the data model can include information that uses incoming real-time data to predict temperate deviations outside of the user's normal temperature pattern as represented by the baseline temperature model. Data that is received can be fed into the data model in real-time. The data model can account for the normal variation pattern exhibited by the human body. For example, it is known that a person's body temperate is higher during waking hours and lower during a period of sleep or rest. The system can consider other factors that also contribute to a person's specific body temperature at any given moment, such as activity level, ambient temperature, alcohol intake, etc. Historical data for an individual user can also be considered in the model.

With the body temperature baseline available, subsequent data feeds collected from the body temperature sensing chip and the application can be reviewed by the server and database connected via the IP switch. Based on the review, the server can be configured with a predetermined protocol to determine if an SMS or VoIP communication should be sent to the user and/or the employer or host of the user. The IP switch preferably includes at least one server and at least one voice-enabled Internet gateway, which is connected to the Internet. The IP switch, and all parts of the IP switch, can be installed with proprietorially written software that operates the protocols and databases described in more detail herein.

In addition to near field communication (NFC), the temperature sensing chip can also communicate via Bluetooth® or other proximate or nearby communication protocol to the application. The temperature sensing chip can be part of a wearable device, such as a bracelet, wristwatch, or smartwatch that can be battery or quartz powered and can continuously report temperature data to the application on the end user's smartphone or mobile device, which can then relay the data to a server. Alternatively, the wearable device can be a smartwatch or other computerized device that can operate the application and communicate directly with a server, such as through cellular or Wi-Fi communications. The temperature sensing chip preferably includes an NFC or Bluetooth® module built-in that is capable of broadcasting data to the application in the mobile device when the wearable device and mobile device are in close proximity. Alternatively, a small, thin chip and NFC or Bluetooth® antenna can be installed on an existing wristwatch, smartwatch or bracelet to thereby enable the wristwatch, smartwatch and bracelet to broadcast biometric data to the coordinating applications, structures and systems of the invention.

In addition to body temperature, the application can also collect other relevant data, such as a location where the body temperature was measured and identifiers of the person whose body temperature is being sensed. The location can be provided by a GPS circuit or RFID chip included in a wearable device in which the body temperature sensors are installed to detect body temperature. An identifier is preferably a value that is stored on the wearable device and/or the mobile device associated with the wearable device and is uniquely associated with the user of the wearable device/mobile device so that the body temperature data provided by the wearable device from the body temperature sensor can be associated with that user. After the application has collected the body temperature data from the wearable device using the application communication protocol, the application can be configured to pass the data to an IP switch that passes the data to a database or a cloud server, or on one or more servers. The body temperature data can be collected over an adjustable time period, such as a second, multiple seconds, a minute, multiple minutes, an hour, multiple hours, a day, multiple days, a week, multiple weeks or a month, and can be used to create a body temperature baseline for the user.

### Wearable Unit and System Hardware

Embodiments include a wearable unit for an end user that is fitted with a blood alcohol concentration sensing or measuring chip that incorporates a Bluetooth® or Nearfield Communication (NFC) module built-in for communicating with a mobile device. The wearable device and smartphone can broadcast collected data through an IP switch to a database and associated servers. The data obtained can be incorporated into a continuously updated and predictive data model that calculates and extrapolates the end user's blood alcohol content. The predictive data model can project what the end user's BAC will be within a period of time (e.g., 15 to 120 minutes). If one of the model curves projects a BAC above a contractually specified limit, the system can send a report to a receiving party, which may include the end user's employer, group-leader, insurance carrier, probation officer or any party subject to an agreement signed and consented to by the end user. The receiving party is further provided with the end user's BAC level in addition to the end user's current GPS location.

While acquiring the blood alcohol content of the end user and constructing predictive models of the acquired data, the system can step through a receiving party's predetermined and specified set of actions depending on the BAC level predicted. The set of actions are database driven and include an arbitrary number of decision points. The actions can include a robust set of communication methods through a proprietary application provided by the present invention, the sending of an SMS message, iMessage, Android message, or VoIP IVR phone call to the receiving party. The system can also interface with the receiving party's own systems and structures to initiate customized actions such as locking out key card credentials, communicating with third-party service providers to send personnel such as taxi cabs, ride-share services, limousine services, helicopter services or tow truck operators to assist the end user at their GPS location. The system can also place the end user in direct VoIP contact with designated medical personnel. The predictive models utilized for the set of actions described above may be gradient boosted models, linear regression models, polynomial regression models, linear/polynomial regression models, naïve Bayes models, or other recognized equivalents well-known in the art. The system can process the obtained BAC data in accordance with the published guidelines from the National Institutes of Health (NIH) and the Centers for Disease Control (CDC) regarding alcohol intoxication.

The present invention may include the use and placement of a Radio Frequency Identification (RFID) chip which can be used to allow or deny access of the end user to privileges specified by the receiving party such as key card entry, overall entry into the receiving party's building, entry into the receiving party's property, entry into the receiving party's company property, and all equivalent forms and modes of access as agreed upon in the signed agreement between the receiving party, end user and all intermediaries involved.

The proprietary application of the present invention can be installed in the end user's smartphone or other equivalent device and function in conjunction with a wearable device. In aspects, the wearable device and smartphone are capable of communicating with each other for data collection, data exchange, data reporting, and dissemination of information throughout the devices and systems of the present invention.

In embodiments, the application receives biometric data from the end user and transmits and reports that data over the Internet to a VoIP-enabled telecommunications switch, which comprises at least one server and at least one voice-enabled Internet gateway which has proprietary program and database(s) installed and is connected to the Internet.

For example, the employer may choose a protocol that says for the IP switch to notify the employee, who has a deviation in blood alcohol concentration, to answer questions coming from the IP switch. These questions might include, for example, asking if the employee is drinking alcohol above a preset limit.

The IP switch can, according to a protocol, ask the end user to text a “1” to the IP switch if they have drunk or ingested less than one alcoholic beverage in the last hour, or text a “2” to the IP switch if the subject has consumed more than two alcoholic beverages in the last hour.

Depending on how the user replies via an SMS (or equivalent) reporting of their use of alcohol, the IP switch can then look to the database and find the receiving party's protocol, thereby the IP Switch can prompt the receiving party to ask the end user to not move from their current location and allow the receiving party to send a car and driver (or equivalent) to pick up the end user and send a flatbed truck to simultaneously retrieve the end user's vehicle.

The IP switch can work through a litany of questions and determine what instruction the receiving party wants delivered to the end user.

For example, in the case of a severe spike in the blood alcohol concentration of an employee, the protocol of the employer can be to tell the employee that their work contract requires that the employee remain where they are and allow the employer to send a car to pick up the employee.

The IP switch can further ask the employee if they received the message that the employer wants the employee to remain exactly where they are physically located.

The IP switch can then send an SMS message (or equivalent) and ask the employee to text a “1” to the IP switch if they received a message about not leaving their current location and text a “2” to the IP switch if they did not receive the message.

If the employee then texts a “2” indicating they did not receive the SMS, then the IP switch can initiate a VoIP call to the cell phone of the employee as recorded in their company file.

This telephone number for the end user is uploaded to the IP switch and into the database of the operator of the IP switch at the time the end user registered their account and created their user name (i.e., user ID) and password using the proprietary application of the present invention.

### Methods of Use

A similar protocol can be applied to the intended purchaser of auto insurance. A member of this group can be offered a lower rate for the purchase of auto insurance if they agree to wear the wearable device for a specified time interval per day. The wearable device can be programmed to alert the seller of the insurance when the insurance buyer (i.e., end user) has stopped reporting data containing their blood alcohol concentration on the network.

A similar protocol can be applied to a probationer after a conviction for driving under the influence. The IP Switch, in conjunction with the blood alcohol concentration measuring chip or device, can be programmed to alert the probation department that the probationer has stopped reporting data containing their blood alcohol concentration. In this case, the IP Switch could alert the probationer that they are not reporting blood alcohol concentration on the network and that in a stated number of minutes or hours, the probationer will be in violation of their probation, which could lead to a warrant for their arrest being issued and a revocation of their probation. In this case, the IP switch can send an SMS message (or equivalent) and ask the probationer to text a “1” to the IP switch if they received a message about possibly being in violation of probation for not reporting their blood alcohol concentration on the network and text a “2” to the IP switch if they did not receive the message.

If the employee then texts a “2” indicating they did not receive the SMS, then the IP switch can initiate a VoIP call to the cell phone of the probationer in their application for probation.

Thereafter, the server can track the responses of the user so that the manager of a group will know if the user is following the protocol for a person with deviations in blood alcohol concentration.

The receiving party (i.e., employer, insurance carrier, insurance agent, probation officer, and anyone specified as a receiving party in the end user's agreement) can modify communication preferences for a particular use. For example, instead of requiring a response of a “1” or a “2,” the receiving party can specify alphabetic responses (e.g., “Y” or “N” or “A” or “B”). The alphabetic responses may be in lower case, upper case, mixed case, or not constrained by syntax. Further, other alphabets are also contemplated for use and are not limited to English or Latin-script alphabets. The receiving party can alternatively require a numeric code such as “12345” or any equivalent specified and/or desired by the receiving party. Further, the numerals programmed into the systems of the present invention may be of any numeric system and are not limited to Roman or Arabic numerals. The receiving party could also specify responses in words and sentences such as “one drink,” “two drinks,” “two drinks or more,” or any other equivalent form of communication that conveys the desired syntax specified by the receiving party regarding an end user's status and response without being limited primarily to the English language.

In further embodiments of the present invention, the blood alcohol content sensor may sample a small quantity (microliters) of sweat and perform an analysis of blood alcohol content every five minutes. In additional embodiments, the blood alcohol content sensor may sample a small quantity of sweat and perform an analysis of blood alcohol content every 5 seconds, every 10 seconds, every 15 seconds, every 20 seconds, every 25 seconds, every 30 seconds, every 45 seconds, every minute, every 90 seconds, every two minutes, every three minutes, every four minutes, every six minutes, every seven minutes, every eight minutes, every nine minutes, every ten minutes, every 12 minutes, every 15 minutes, every 16 minutes, every 18 minutes, every 20 minutes, every 23 minutes, every 25 minutes, every 27 minutes, every 30 minutes, every 32 minutes, every 35 minutes, every 37 minutes, every 40 minutes, every 43 minutes, every 45 minutes, every 50 minutes, every 52 minutes, every 55 minutes, every hour, every 65 minutes, every 70 minutes, every 75 minutes, every 80 minutes, every 90 minutes, every 100 minutes, every two hours, every 150 minutes, every three hours, every 210 minutes, every four hours, every five hours, every six hours, every seven hours, every eight hours, every nine hours, every ten hours or more.

In embodiments of the present invention, the blood alcohol content sensor may sample a small quantity (microliters) of sweat and perform an analysis of blood alcohol content at least every five minutes. In additional embodiments, the blood alcohol content sensor may sample a small quantity of sweat and perform an analysis of blood alcohol content at least every 5 seconds, at least every 10 seconds, at least every 15 seconds, at least every 20 seconds, at least every 25 seconds, at least every 30 seconds, at least every 45 seconds, at least every minute, at least every 90 seconds, at least every two minutes, at least every three minutes, at least every four minutes, at least every six minutes, at least every seven minutes, at least every eight minutes, at least every nine minutes, at least every ten minutes, at least every 12 minutes, at least every 15 minutes, at least every 16 minutes, at least every 18 minutes, at least every 20 minutes, at least every 23 minutes, at least every 25 minutes, at least every 27 minutes, at least every 30 minutes, at least every 32 minutes, at least every 35 minutes, at least every 37 minutes, at least every 40 minutes, at least every 43 minutes, at least every 45 minutes, at least every 50 minutes, at least every 52 minutes, at least every 55 minutes, at least every hour, at least every 65 minutes, at least every 70 minutes, at least every 75 minutes, at least every 80 minutes, at least every 90 minutes, at least every 100 minutes, at least every two hours, at least every 150 minutes, at least every three hours, at least every 210 minutes, at least every four hours, at least every five hours, at least every six hours, at least every seven hours, at least every eight hours, at least every nine hours, at least every ten hours or more.

In additional embodiments of the present invention, the blood alcohol content sensor may sample a small quantity of sweat and perform an analysis of blood alcohol content about every 5 seconds, about every 10 seconds, about every 15 seconds, about every 20 seconds, about every 25 seconds, about every 30 seconds, about every 45 seconds, about every minute, about every 90 seconds, about every two minutes, about every three minutes, about every four minutes, about every six minutes, about every seven minutes, about every eight minutes, about every nine minutes, about every ten minutes, about every 12 minutes, about every 15 minutes, about every 16 minutes, about every 18 minutes, about every 20 minutes, about every 23 minutes, about every 25 minutes, about every 27 minutes, about every 30 minutes, about every 32 minutes, about every 35 minutes, about every 37 minutes, about every 40 minutes, about every 43 minutes, about every 45 minutes, about every 50 minutes, about every 52 minutes, about every 55 minutes, about every hour, about every 65 minutes, about every 70 minutes, about every 75 minutes, about every 80 minutes, about every 90 minutes, about every 100 minutes, about every two hours, about every 150 minutes, about every three hours, about every 210 minutes, about every four hours, about every five hours, about every six hours, about every seven hours, about every eight hours, about every nine hours, about every ten hours or more.

In further embodiments of the present invention, the blood alcohol content sensor may sample a quantity of sweat from the end user in quantities of picoliters, nanoliters, microliters, milliliters or any suitable quantity recognized by the art.

In additional embodiments of the present invention, the blood alcohol content sensor detects blood alcohol content by comparing a current temperature of an end user with a baseline temperature of the end user.

In further embodiments of the present invention, the blood alcohol content sensor detects blood alcohol content by comparing a current temperature of an end user with a baseline temperature of the end user every millisecond, every 2 milliseconds, every 3 milliseconds, every 4 milliseconds, every 5 milliseconds, every 6 milliseconds, every 7 milliseconds, every 8 milliseconds, every 9 milliseconds, every 10 milliseconds, every 12 milliseconds, every 15 milliseconds, every 17 milliseconds, every 20 milliseconds, every 23 milliseconds, every 25 milliseconds, every 27 milliseconds, every 30 milliseconds, every 35 milliseconds, every 40 milliseconds, every 45 milliseconds, every 50 milliseconds, every 55 milliseconds, every 60 milliseconds, every 65 milliseconds, every 70 milliseconds, every 75 milliseconds, every 80 milliseconds, every 90 milliseconds, every 100 milliseconds, every 110 milliseconds, every 125 milliseconds, every 150 milliseconds, every 175 milliseconds, every 200 milliseconds, every 225 milliseconds, every 250 milliseconds, every 275 milliseconds, every 300 milliseconds, every 325 milliseconds, every 350 milliseconds, every 375 milliseconds, every 400 milliseconds, every 425 milliseconds, every 450 milliseconds, every 475 milliseconds, every 500 milliseconds, every 525 milliseconds, every 550 milliseconds, every 575 milliseconds, every 600 milliseconds, every 625 milliseconds, every 650 milliseconds, every 675 milliseconds, every 700 milliseconds, every 725 milliseconds, every 750 milliseconds, every 775 milliseconds, every 800 milliseconds, every 825 milliseconds, every 850 milliseconds, every 900 milliseconds, every 950 milliseconds, every second, every two seconds, every three seconds, every four seconds, every five seconds, every six seconds, every seven seconds, every eight seconds, every nine seconds, every ten seconds, every 11 seconds, every 12 seconds, every 13 seconds, every 14 seconds, every 15 seconds, every 16 seconds, every 17 seconds, every 18 seconds, every 19 seconds, every 20 seconds, every 25 seconds, every 30 seconds, every 35 seconds, every 40 seconds, every 45 seconds, every 50 seconds, every 55 seconds, every 60 seconds, every 65 seconds, every 70 seconds, every 75 seconds, every 80 seconds, every 85 seconds, every 90 seconds, every 95 seconds, every 100 seconds, every 110 seconds, every minute, every two minutes, every three minutes, every four minutes, every five minutes, every six minutes, every seven minutes, every eight minutes, every nine minutes, every ten minutes, every 11 minutes, every 12 minutes, every 13 minutes, every 14 minutes, every 15 minutes, every 20 minutes, every 23 minutes, every 25 minutes, every 30 minutes, every 35 minutes, every 40 minutes, every 45 minutes, every 50 minutes, every 55 minutes, every hour, every 65 minutes, every 70 minutes, every 75 minutes, every 80 minutes, every 85 minutes, every 90 minutes, every 95 minutes, every 100 minutes, every 110 minutes, every two hours, every 130 minutes, every 150 minutes, every 175 minutes, every three hours, every 200 minutes, every 225 minutes, every four hours, every five hours, every six hours, every seven hours, every eight hours, every nine hours, every ten hours, every 11 hours, every 12 hours, every 13 hours, every 14 hours, every 15 hours, every 16 hours, every 17 hours, every 18 hours, every 19 hours, every 20 hours, every 21 hours, every 22 hours, every 23 hours, every day, twice per day, three times per day, four times per day, five times per day, six times per day, seven times per day, eight times per day, nine times per day, ten times per day, 11 times per day or more, once per week, twice per week, three times per week, four times per week, five times per week, six times per week, seven times per week or more.

In additional embodiments of the present invention, the blood alcohol content sensor detects blood alcohol content by comparing a current temperature of an end user with a baseline temperature of the end user about every millisecond, about every 2 milliseconds, about every 3 milliseconds, about every 4 milliseconds, about every 5 milliseconds, about every 6 milliseconds, about every 7 milliseconds, about every 8 milliseconds, about every 9 milliseconds, about every 10 milliseconds, about every 12 milliseconds, about every 15 milliseconds, about every 17 milliseconds, about every 20 milliseconds, about every 23 milliseconds, about every 25 milliseconds, about every 27 milliseconds, about every 30 milliseconds, about every 35 milliseconds, about every 40 milliseconds, about every 45 milliseconds, about every 50 milliseconds, about every 55 milliseconds, about every 60 milliseconds, about every 65 milliseconds, about every 70 milliseconds, about every 75 milliseconds, about every 80 milliseconds, about every 90 milliseconds, about every 100 milliseconds, about every 110 milliseconds, about every 125 milliseconds, about every 150 milliseconds, about every 175 milliseconds, about every 200 milliseconds, about every 225 milliseconds, about every 250 milliseconds, about every 275 milliseconds, about every 300 milliseconds, about every 325 milliseconds, about every 350 milliseconds, about every 375 milliseconds, about every 400 milliseconds, about every 425 milliseconds, about every 450 milliseconds, about every 475 milliseconds, about every 500 milliseconds, about every 525 milliseconds, about every 550 milliseconds, about every 575 milliseconds, about every 600 milliseconds, about every 625 milliseconds, about every 650 milliseconds, about every 675 milliseconds, about every 700 milliseconds, about every 725 milliseconds, about every 750 milliseconds, about every 775 milliseconds, about every 800 milliseconds, about every 825 milliseconds, about every 850 milliseconds, about every 900 milliseconds, about every 950 milliseconds, about every second, about every two seconds, about every three seconds, about every four seconds, about every five seconds, about every six seconds, about every seven seconds, about every eight seconds, about every nine seconds, about every ten seconds, about every 11 seconds, about every 12 seconds, about every 13 seconds, about every 14 seconds, about every 15 seconds, about every 16 seconds, about every 17 seconds, about every 18 seconds, about every 19 seconds, about every 20 seconds, about every 25 seconds, about every 30 seconds, about every 35 seconds, about every 40 seconds, about every 45 seconds, about every 50 seconds, about every 55 seconds, about every 60 seconds, about every 65 seconds, about every 70 seconds, about every 75 seconds, about every 80 seconds, about every 85 seconds, about every 90 seconds, about every 95 seconds, about every 100 seconds, about every 110 seconds, about every minute, about every two minutes, about every three minutes, about every four minutes, about every five minutes, about every six minutes, about every seven minutes, about every eight minutes, about every nine minutes, about every ten minutes, about every 11 minutes, about every 12 minutes, about every 13 minutes, about every 14 minutes, about every 15 minutes, about every 20 minutes, about every 23 minutes, about every 25 minutes, about every 30 minutes, about every 35 minutes, about every 40 minutes, about every 45 minutes, about every 50 minutes, about every 55 minutes, about every hour, about every 65 minutes, about every 70 minutes, about every 75 minutes, about every 80 minutes, about every 85 minutes, about every 90 minutes, about every 95 minutes, about every 100 minutes, about every 110 minutes, about every two hours, about every 130 minutes, about every 150 minutes, about every 175 minutes, about every three hours, about every 200 minutes, about every 225 minutes, about every four hours, about every five hours, about every six hours, about every seven hours, about every eight hours, about every nine hours, about every ten hours, about every 11 hours, about every 12 hours, about every 13 hours, about every 14 hours, about every 15 hours, about every 16 hours, about every 17 hours, about every 18 hours, about every 19 hours, about every 20 hours, about every 21 hours, about every 22 hours, about every 23 hours, about every day, about twice per day, about three times per day, about four times per day, about five times per day, about six times per day, about seven times per day, about eight times per day, about nine times per day, about ten times per day, about 11 times per day or more, about once per week, about twice per week, about three times per week, about four times per week, about five times per week, about six times per week, about seven times per week or more.

In further embodiments of the present invention, the blood alcohol content sensor detects blood alcohol content by comparing a current temperature of an end user with a baseline temperature of the end user at least every millisecond, at least every 2 milliseconds, at least every 3 milliseconds, at least every 4 milliseconds, at least every 5 milliseconds, at least every 6 milliseconds, at least every 7 milliseconds, at least every 8 milliseconds, at least every 9 milliseconds, at least every 10 milliseconds, at least every 12 milliseconds, at least every 15 milliseconds, at least every 17 milliseconds, at least every 20 milliseconds, at least every 23 milliseconds, at least every 25 milliseconds, at least every 27 milliseconds, at least every 30 milliseconds, at least every 35 milliseconds, at least every 40 milliseconds, at least every 45 milliseconds, at least every 50 milliseconds, at least every 55 milliseconds, at least every 60 milliseconds, at least every 65 milliseconds, at least every 70 milliseconds, at least every 75 milliseconds, at least every 80 milliseconds, at least every 90 milliseconds, at least every 100 milliseconds, at least every 110 milliseconds, at least every 125 milliseconds, at least every 150 milliseconds, at least every 175 milliseconds, at least every 200 milliseconds, at least every 225 milliseconds, at least every 250 milliseconds, at least every 275 milliseconds, at least every 300 milliseconds, at least every 325 milliseconds, at least every 350 milliseconds, at least every 375 milliseconds, at least every 400 milliseconds, at least every 425 milliseconds, at least every 450 milliseconds, at least every 475 milliseconds, at least every 500 milliseconds, at least every 525 milliseconds, at least every 550 milliseconds, at least every 575 milliseconds, at least every 600 milliseconds, at least every 625 milliseconds, at least every 650 milliseconds, at least every 675 milliseconds, at least every 700 milliseconds, at least every 725 milliseconds, at least every 750 milliseconds, at least every 775 milliseconds, at least every 800 milliseconds, at least every 825 milliseconds, at least every 850 milliseconds, at least every 900 milliseconds, at least every 950 milliseconds, at least every second, at least every two seconds, at least every three seconds, at least every four seconds, at least every five seconds, at least every six seconds, at least every seven seconds, at least every eight seconds, at least every nine seconds, at least every ten seconds, at least every 11 seconds, at least every 12 seconds, at least every 13 seconds, at least every 14 seconds, at least every 15 seconds, at least every 16 seconds, at least every 17 seconds, at least every 18 seconds, at least every 19 seconds, at least every 20 seconds, at least every 25 seconds, at least every 30 seconds, at least every 35 seconds, at least every 40 seconds, at least every 45 seconds, at least every 50 seconds, at least every 55 seconds, at least every 60 seconds, at least every 65 seconds, at least every 70 seconds, at least every 75 seconds, at least every 80 seconds, at least every 85 seconds, at least every 90 seconds, at least every 95 seconds, at least every 100 seconds, at least every 110 seconds, at least every minute, at least every two minutes, at least every three minutes, at least every four minutes, at least every five minutes, at least every six minutes, at least every seven minutes, at least every eight minutes, at least every nine minutes, at least every ten minutes, at least every 11 minutes, at least every 12 minutes, at least every 13 minutes, at least every 14 minutes, at least every 15 minutes, at least every 20 minutes, at least every 23 minutes, at least every 25 minutes, at least every 30 minutes, at least every 35 minutes, at least every 40 minutes, at least every 45 minutes, at least every 50 minutes, at least every 55 minutes, at least every hour, at least every 65 minutes, at least every 70 minutes, at least every 75 minutes, at least every 80 minutes, at least every 85 minutes, at least every 90 minutes, at least every 95 minutes, at least every 100 minutes, at least every 110 minutes, at least every two hours, at least every 130 minutes, at least every 150 minutes, at least every 175 minutes, at least every three hours, at least every 200 minutes, at least every 225 minutes, at least every four hours, at least every five hours, at least every six hours, at least every seven hours, at least every eight hours, at least every nine hours, at least every ten hours, at least every 11 hours, at least every 12 hours, at least every 13 hours, at least every 14 hours, at least every 15 hours, at least every 16 hours, at least every 17 hours, at least every 18 hours, at least every 19 hours, at least every 20 hours, at least every 21 hours, at least every 22 hours, at least every 23 hours, at least every day, at least twice per day, at least three times per day, at least four times per day, at least five times per day, at least six times per day, at least seven times per day, at least eight times per day, at least nine times per day, at least ten times per day, at least 11 times per day or more, at least once per week, at least twice per week, at least three times per week, at least four times per week, at least five times per week, at least six times per week, at least seven times per week or more.

In embodiments of the invention the blood alcohol content sensor comprises one or more of a chip, a microprocessor, a CPU, a sensor, a thermocouple, a thermometer, a pressure sensor, a temperature sensor, a touch sensor, a light sensor, a motion sensor, a vibration sensor, a gyroscope, a tilt sensor, an accelerometer, or any other equivalent recognized and well-known in the art.

In embodiments of the present invention, the predictive models utilized to extrapolate and communicate blood alcohol content are selected from a group comprising a gradient boosted model, a linear regression model, a polynomial regression model, a linear/polynomial regression model, a naïve Bayes model, or any other art-recognized and well-known equivalent known in the art.

According to embodiments of the present invention, an employer can monitor employees regardless of location to detect significant spikes or changes in the body temperature of their employees who may be coming and going to the employer's place of business. Similarly, a host, such as a hotel operator, resort operator, airline operator, landlord, temporary landlord (e.g., AirBnb® owner or operator) or cruise ship director, can monitor guests regardless of location to detect significant spikes or changes in the body temperature of the guests who may be coming and going to the host's venue. In one implementation, the monitoring and detection can include an Internet Protocol (IP) telecommunications switch and a proprietary application installed on a smartphone or other mobile device. The application can be configured to collect the body temperature data from a temperature sensing chip or other body temperature measuring element that is installed next to the skin, such as on the wrist, via a communication protocol such as near field communication module (NFC), Bluetooth, Wi-Fi, or other protocol or communication system or information dissemination system capable of sending data between a wearable device and a mobile device.

### Operating Environment

The system is typically comprised of a central server that is connected by a data network to a user's (e.g., a user's) computer, phone or tablet. The central server can be comprised of one or more computers connected to one or more mass storage devices. The precise architecture of the central server does not limit the claimed invention. Further, the user's computer can be a laptop or desktop type of personal computer. It can also be a cell phone, smartphone or other handheld device, including a tablet. The precise form factor of the user's computer does not limit the claimed invention. Examples of well-known computing systems, environments, and/or configurations that may be suitable for use with the invention include, but are not limited to, personal computers, server computers, hand held, laptop or mobile computer or communications devices such as cell phones and PDA's, multiprocessor systems, microprocessor-based systems, set top boxes, programmable consumer electronics, network PCs, minicomputers, microcomputers, mainframe computers, distributed computing environments that include any of the above systems or devices, and the like. The precise form factor of the user's computer does not limit the claimed invention. In one embodiment, the user's computer is omitted, and instead a separate computing functionality provided that works with the central server. In this case, a user would log into the server from another computer and access the system through a user environment.

The user environment can be housed in the central server or operatively connected to it. Further, the user can receive from and transmit data to the central server by means of the Internet, whereby the user accesses an account using an Internet web-browser and browser displays an interactive web page operatively connected to the central server. The central server transmits and receives data in response to data and commands transmitted from the browser in response to the customer's actuation of the browser user interface. Some steps of the invention may be performed on the user's computer and interim results transmitted to a server. These interim results may be processed at the server and final results passed back to the user.

The methods described herein can be executed on a computer system, generally comprised of a central processing unit (CPU) that is operatively connected to a memory device, data input and output circuitry (I/O) and computer data network communication circuitry. Computer code executed by the CPU can take data received by the data communication circuitry and store it in the memory device. In addition, the CPU can take data from the I/O circuitry and store it in the memory device. Further, the CPU can take data from a memory device and output it through the I/O circuitry or the data communication circuitry. The data stored in memory may be further recalled from the memory device, further processed or modified by the CPU in the manner described herein and restored in the same memory device or a different memory device operatively connected to the CPU including by means of the data network circuitry. The memory device can be any kind of data storage circuit or magnetic storage or optical device, including a hard disk, optical disk or solid state memory. The I/O devices can include a display screen, loudspeakers, microphone and a movable mouse that indicate to the computer the relative location of a cursor position on the display and one or more buttons that can be actuated to indicate a command.

The computer can display on the display screen operatively connected to the I/O circuitry the appearance of a user interface. Various shapes, text and other graphical forms are displayed on the screen as a result of the computer generating data that causes the pixels comprising the display screen customer's actuation of the browser user interface. Some steps of the invention can be performed on the user's computer and interim results transmitted to a server. These interim results can be processed at the server and final results passed back to the user.

The invention may also be entirely executed on one or more servers. A server may be a computer comprised of a central processing unit with a mass storage device and a network connection. In addition, a server can include multiple of such computers connected together with a data network or other data transfer connection, or, multiple computers on a network with network accessed storage, in a manner that provides such functionality as a group. Practitioners of ordinary skill will recognize that functions that are accomplished on one server may be partitioned and accomplished on multiple servers that are operatively connected by a computer network by means of appropriate inter process communication. In addition, the access of the website can be by means of an Internet browser accessing a secure or public page or by means of a client program running on a local computer that is connected over a computer network to the server. A data message and data upload or download can be delivered over the Internet using typical protocols, including TCP/IP, HTTP, TCP, UDP, SMTP, RPC, FTP or other kinds of data communication protocols that permit processes running on two remote computers to exchange information by means of digital network communication. As a result, a data message can be a data packet transmitted from or received by a computer containing a destination network address, a destination process or application identifier, and data values that can be parsed at the destination computer located at the destination network address by the destination application in order that the relevant data values are extracted and used by the destination application. The precise architecture of the central server does not limit the claimed invention. In addition, the data network may operate with several levels, such that the user's computer is connected through a firewall to one server, which routes communications to another server that executes the disclosed methods.

Computer program logic implementing all or part of the functionality previously described herein may be embodied in various forms, including, but in no way limited to, a source code form, a computer executable form, and various intermediate forms (e.g., forms generated by an assembler, compiler, linker, or locator.) Source code may include a series of computer program instructions implemented in any of various programming languages (e.g., an object code, an assembly language, or a high-level language such as C, C-HF, C#, Action Script, PHP, ECMAScript, JavaScript, JAVA, or 5 HTML) for use with various operating systems or operating environments. The source code may define and use various data structures and communication messages. The source code may be in a computer executable form (e.g., via an interpreter), or the source code may be converted (e.g., via a translator, assembler, or compiler) into a computer executable form.

The invention may be described in the general context of computer-executable instructions, such as program modules, being executed by a computer. Generally, program modules include routines, programs, objects, components, data structures, etc., that perform particular tasks or implement particular abstract data types. The computer program and data may be fixed in any form (e.g., source code form, computer executable form, or an intermediate form) either permanently or transitorily in a tangible storage medium, such as a semiconductor memory device (e.g., a RAM, ROM, PROM, EEPROM, or flash-programmable RAM), a magnetic memory device (e.g., a diskette or fixed hard disk), an optical memory device (e.g., a CD-ROM or DVD), a PC card (e.g., PCMCIA card), or other memory device. The computer program and data may be fixed in any form in a signal that is transmittable to a computer using any of various communication technologies, including, but in no way limited to, analog technologies, digital technologies, optical technologies, wireless technologies, networking technologies, and internetworking technologies. The computer program and data may be distributed in any form as a removable storage medium with accompanying printed or electronic documentation (e.g., shrink-wrapped software or a magnetic tape), preloaded with a computer system (e.g., on system ROM or fixed disk), or distributed from a server or electronic bulletin board over the communication system (e.g., the Internet or World Wide Web.) It is appreciated that any of the software components of the present invention may, if desired, be implemented in ROM (read-only memory) form. The software components may, generally, be implemented in hardware, if desired, using conventional techniques.

The invention may also be practiced in distributed computing environments where tasks are performed by remote processing devices that are linked through a communications network. In a distributed computing environment, program modules may be located in both local and remote computer storage media including memory storage devices. Practitioners of ordinary skill will recognize that the invention may be executed on one or more computer processors that are linked using a data network, including, for example, the Internet. In another embodiment, different steps of the process can be executed by one or more computers and storage devices geographically separated but connected by a data network in a manner so that they operate together to execute the process steps. In one embodiment, a user's computer can run an application that causes the user's computer to transmit a stream of one or more data packets across a data network to a second computer, referred to here as a server. The server, in turn, may be connected to one or more mass data storage devices where the database is stored. The server can execute a program that receives the transmitted packet and interpret the transmitted data packets in order to extract database query information. The server can then execute the remaining steps of the invention by means of accessing the mass storage devices to derive the desired result of the query. Alternatively, the server can transmit the query information to another computer that is connected to the mass storage devices, and that computer can execute the invention to derive the desired result. The result can then be transmitted back to the user's computer by means of another stream of one or more data packets appropriately addressed to the user's computer. In one embodiment, the relational database may be housed in one or more operatively connected servers operatively connected to computer memory, for example, disk drives. In yet another embodiment, the initialization of the relational database may be prepared on the set of servers and the interaction with the user's computer occur at a different place in the overall process.

It should be noted that the flow diagrams are used herein to demonstrate various aspects of the invention, and should not be construed to limit the invention to any particular logic flow or logic implementation. The described logic may be partitioned into different logic blocks (e.g., programs, modules, functions, or subroutines) without changing the overall results or otherwise departing from the true scope of the invention. Oftentimes, logic elements may be added, modified, omitted, performed in a different order, or implemented using different logic constructs (e.g., logic gates, looping primitives, conditional logic, and other logic constructs) without changing the overall results or otherwise departing from the true scope of the invention.

### EXAMPLES

### Example 1

### Monitoring an Employee's BAC

An employee (“end user”) consents to and signs an agreement specifying that through the wearable device, proprietary application and systems of the present invention, they will continuously report their blood alcohol content instantly and automatically via the wearable device, proprietary application and systems of the present invention. The agreed-upon and specified threshold for the employee's BAC is 0.08%.

Thirty-five days after signing the agreement and adopting the wearable device, proprietary application and systems of the present invention, the employee receives an SMS alert on their way to work which asks the employee to tap their smartphone against the wearable device of the present invention. The employee does so, and through the proprietary application of the present invention, it is reported to the employer that the employee's blood alcohol content is 0.11%, surpassing the 0.08% threshold specified by the employer-employee agreement. Upon obtaining this data, the proprietary application of the present invention initiates a protocol which locks the employee out of the employer's building, temporarily voiding the employee's key card credentials. In addition, the employee receives an SMS message asking them to pull over their vehicle and await a ride-share service which will pick them up from their current location and deliver the employee back to their place of residence. Meanwhile, a tow truck service is automatically alerted and delivers a flat-bed truck to retrieve the employee's vehicle and return it to the employee's residence.

### Example 2

### Monitoring BAC During Probation

A person convicted of a DUI is currently on probation. As part of their probationary agreement, the person must consistently report their BAC level to a probation officer via the devices, systems, and proprietary applications of the present invention. On the 20^{th} day of their probation, at 3:30 PM local time, this person receives an Android message requesting that they bring their smartphone within close contact of their smartwatch containing the proprietary application of the present invention so their BAC level may be obtained and instantly reported to their probation officer. This person does so, obtaining a BAC result of 0.0% which is well within the terms of their probation and therefore no disciplinary action is required by the probation officer.

### Example 3

### Monitoring BAC for Auto Insurance

As part of their auto insurance contract, an auto insurance customer must wear a bracelet with the proprietary application of the present invention installed whenever they are inside the vehicle specified on their auto insurance policy. When this auto insurance customer wears the bracelet with the proprietary application of the present invention installed, the auto insurance agent who sold the auto insurance to the customer automatically receives continuously updated information regarding the customer's body temperature, GPS location, and BAC level. On the 100^{th} day of their auto insurance policy, the auto insurance agent receives an alert that their customer has a contemporaneous BAC of 0.10%. This is in violation of the stipulated agreement that the auto insurance customer consented to, and therefore their auto insurance policy is immediately and effectively void.

### Example 4

### Monitoring an Airline Employee

An airline pilot has agreed with their employer to register with the proprietary application of the instant invention and agreed to wear a smartwatch which will continuously communicate their BAC level during working hours. Three weeks after the agreement, as the airline pilot is driving toward their assigned airport, the pilot receives an iMessage asking them to tap their smartphone against their smartwatch so as to obtain their contemporaneous BAC data.

Upon tapping their smartphone to their smartwatch, the airline pilot is surprised to find that their BAC level registers at 0.02% BAC. This information is immediately delivered to all receiving parties, including the airline pilot's direct supervisor. This BAC level is in violation of the stipulated agreement between the airline pilot and the receiving parties, and as such, the airline pilot immediately receives an iMessage stating that they should pull over their vehicle and await a rideshare service. The airline pilot does as instructed, awaiting for arrival of the rideshare service as a tow truck service is automatically alerted and in transit to pick up the airline pilot's vehicle and deliver it back to their residence. The airline pilot's credentials are voided for 24 hours, meaning that the airline pilot is unable to bypass airport security if they were to attempt to do so. The airline pilot is picked up by the rideshare service and delivered to their residence on file.

### Example 5

### Monitoring an Employee's BAC (Employer Event)

An employee is attending their employer's holiday party on December 19^{th}. The employee has a fun time interacting with fellow employees but imbibes several alcoholic drinks. The employee makes an ill-advised decision by electing to drive their vehicle home to their residence immediately following the party. Upon entering their vehicle, the employee is alerted on their smartphone to place their smartphone within near-contact of a wrist-worn bracelet with the proprietary application of the present invention installed. The employee obliges and taps their smartphone against their wrist-worn bracelet, and immediately their employer is alerted to the employee's BAC level, which is 0.12%.

Instantly, the employee receives an SMS message asking them to remain at their current location and await a rideshare service. The employee also receives a phone call, where an automated operator instructs them to remain at their current location and await a rideshare service in transit. The employee does as instructed and is picked up by the rideshare service and transported to their residence on file. Shortly thereafter, a tow truck service delivers the employee's vehicle to the same residence on file.

### Example 6

### Predictive BAC Threshold Violation by Professional Athlete

A running back for a National Football League® team is enjoying an in-season rest period with his teammates. On the night before their weekly day off, the running back and several of his teammates visit several bars and clubs. At the conclusion of their night out together, the running back feels okay to drive home, but is tempted in drinking a shot of 100-proof liquor with his teammates before departing. The running back drinks the shot of liquor, departs the bar, and gets into his Mercedes Benz C-Class Cabriolet® convertible. Upon entering his car, he is alerted by his Apple® smartwatch with the following message, “Your predicted BAC is over the limit, please await rideshare service and do not operate your vehicle.”

The running back does as instructed. Within 15 minutes, a rideshare vehicle arrives and picks up the running back and returns him to his home address on company file. Shortly after being picked up, a flatbed tow truck arrives at the site of the running back's parked convertible. A tow truck operator loads the convertible onto the flatbed tow truck and delivers the convertible to the running back's home address on company file.

Certain embodiments of the present invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the present invention to be practiced otherwise than specifically described herein. Accordingly, this invention includes all modifications and equivalents of the subject matter recited in the claims appended hereto as permitted by applicable law. Moreover, any combination of the above-described embodiments in all possible variations thereof is encompassed by the invention unless otherwise indicated herein or otherwise clearly contradicted by context.

Groupings of alternative embodiments, elements, or steps of the present invention are not to be construed as limitations. Each group member may be referred to and claimed individually or in any combination with other group members disclosed herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Unless otherwise indicated, all numbers expressing a characteristic, item, quantity, parameter, property, term, and so forth used in the present specification and claims are to be understood as being modified in all instances by the term “about.” As used herein, the term “about” means that the characteristic, item, quantity, parameter, property, or term so qualified encompasses a range of plus or minus ten percent above and below the value of the stated characteristic, item, quantity, parameter, property, or term. Accordingly, unless indicated to the contrary, the numerical parameters set forth in the specification and attached claims are approximations that may vary. At the very least, and not as an attempt to limit the application of the doctrine of equivalents to the scope of the claims, each numerical indication should at least be construed in light of the number of reported significant digits and by applying ordinary rounding techniques. Notwithstanding that the numerical ranges and values setting forth the broad scope of the invention are approximations, the numerical ranges and values set forth in the specific examples are reported as precisely as possible. Any numerical range or value, however, inherently contains certain errors necessarily resulting from the standard deviation found in their respective testing measurements. Recitation of numerical ranges of values herein is merely intended to serve as a shorthand method of referring individually to each separate numerical value falling within the range. Unless otherwise indicated herein, each individual value of a numerical range is incorporated into the present specification as if it were individually recited herein.

The terms “a,” “an,” “the” and similar referents used in the context of describing the present invention (especially in the context of the following claims) are to be construed to cover both the singular and the plural, unless otherwise indicated herein or clearly contradicted by context. All methods described herein can be performed in any suitable order unless otherwise indicated herein or otherwise clearly contradicted by context. The use of any and all examples, or exemplary language (e.g., “such as”) provided herein is intended merely to better illuminate the present invention and does not pose a limitation on the scope of the invention otherwise claimed. No language in the present specification should be construed as indicating any non-claimed element essential to the practice of the invention.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the present invention so claimed are inherently or expressly described and enabled herein.

Groupings of alternative embodiments, elements, or steps of the present invention are not to be construed as limitations. Each group member may be referred to and claimed individually or in any combination with other group members disclosed herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

All patents, patent publications, and other publications referenced and identified in the present specification are individually and expressly incorporated herein by reference in their entirety for the purpose of describing and disclosing, for example, the compositions and methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

In closing, it is to be understood that although aspects of the present specification are highlighted by referring to specific embodiments, one skilled in the art will readily appreciate that these disclosed embodiments are only illustrative of the principles of the subject matter disclosed herein. Therefore, it should be understood that the disclosed subject matter is in no way limited to a particular methodology, protocol, and/or reagent, etc., described herein. As such, various modifications or changes to or alternative configurations of the disclosed subject matter can be made in accordance with the teachings herein without departing from the spirit of the present specification. Lastly, the terminology used herein is for the purpose of describing particular embodiments only, and is not intended to limit the scope of the present invention, which is defined solely by the claims. Accordingly, the present invention is not limited to that precisely as shown and described.

What is claimed is:

1. A computer-implemented method for monitoring and predicting blood alcohol levels in a subject, the method comprising:
a) detecting a present blood alcohol content using one or more sensors on a wearable device;
b) estimating a future blood alcohol content;
c) reporting the present blood alcohol content and the future blood alcohol content to a server;
d) activating an alert system if the present blood alcohol content and/or the future blood alcohol content is at or above a threshold level.

2. The computer-implemented method of claim 1, further comprising a step of detecting one or more physiological events in the subject.

3. The computer-implemented method of claim 2, wherein the one or more physiological events are selected from eye/pupil activity, heart rate, change in heart rate, bodily movement, temperature, change in temperature, blood pressure, change in blood pressure, respiration rate, level of oxygen saturation, perspiration rate and blood glucose level.

4. The computer-implemented method of claim 3, wherein the one or more physiological events are considered in predicting the future blood alcohol content.

5. The computer-implemented method of claim 1, wherein historical data is considered in predicting the future blood alcohol content.

6. The computer-implemented method of claim 1, wherein the threshold blood alcohol content is 0.08%.

7. The computer-implemented method of claim 1, wherein the step of estimating a future blood alcohol content uses deep learning and/or a neural network.

8. The computer-implemented method of claim 1, wherein the step of estimating a future blood alcohol content uses a data model selected from a linear regression model, a polynomial regression model, a naïve Bayes model and a gradient boosted model.

9. The computer-implemented method of claim 1, wherein the present blood alcohol content is detected transdermally.

10. The computer-implemented method of claim 1, wherein the alert system notifies one or more of a parent, employer, probation officer, insurance company, ride share agent, tow truck operator, group leader or custodial agent.

11. The computer-implemented method of claim 1, wherein the alert system comprises geographical location information.

12. The computer-implemented method of claim 1, wherein the future blood alcohol content is estimated for a future time point, wherein the future time point is between 15 to 120 minutes from a present time.

13. The computer-implemented method of claim 1, further comprising a step of encrypting data related to identity of the subject.

14. A computer-implemented method for evaluating and predicting impairment in a subject, the method comprising:
a) detecting a blood alcohol content using one or more sensors on a wearable device;
b) detecting one or more physiological events using one or more sensors on a wearable device;
c) determining a present level of impairment based on the blood alcohol content and the one or more physiological events;
d) estimating a future level of impairment based on the blood alcohol content and the one or more physiological events;
e) reporting the present level of impairment and future level of impairment to a server;
f) activating an alert system if the present level of impairment and/or the future level of impairment is at or above a threshold level.

15. The computer-implemented method of claim 14, wherein the one or more physiological events are selected from eye/pupil activity, heart rate, change in heart rate, bodily movement, temperature, change in temperature, blood pressure, change in blood pressure, respiration rate, level of oxygen saturation, perspiration rate and blood glucose level.

16. The computer-implemented method of claim 14, wherein historical data is used in the step of estimating a future level of impairment.

17. The computer-implemented method of claim 14, wherein the step of estimating the future level of impairment uses deep learning and/or a neural network.

18. The computer-implemented method of claim 14, wherein the step of estimating the future level of impairment uses a data model selected from a linear regression model, a polynomial regression model, a naïve Bayes model and a gradient boosted model.

19. The computer-implemented method of claim 14, wherein the blood alcohol content is detected transdermally.

20. The computer-implemented method of claim 14, further comprising a step of encrypting data related to identity of the subject.
